(19) United States
(12) Patent Application Publication
(10) Pub. No.: US 2011/0245731 A1
(43) Pub. Date: Oct. 6, 2011

(54) MEDICAL APPARATUS SYSTEM, CAPSULE MEDICAL APPARATUS SYSTEM, AND METHOD OF DISPLAYING POSTURE ITEM OF SUBJECT

(75) Inventors: Atsushi CHIBA, Tokyo (JP); Miho KATAYAMA, Yokohama-shi (JP); Takeshi NISHIYAMA, Tokyo (JP)

(73) Assignee: OLYMPUS MEDICAL SYSTEMS CORP., Tokyo (JP)

(21) Appl. No.: 13/072,149

(22) Filed: Mar. 25, 2011

Related U.S. Application Data
(63) Continuation of application No. PCT/JP2010/062553, filed on Jul. 26, 2010.

(30) Foreign Application Priority Data
Nov. 19, 2009 (JP) 2009-264276

(51) Int. Cl. A61B 5/103

(52) U.S. Cl. 600/587

(57) ABSTRACT

A medical apparatus system includes a medical apparatus that acquires information on a subject; a posture item display unit that displays multiple posture items of the subject in which the medical apparatus acquires the information on the subject; an input unit that inputs a posture item of the subject in which the information on the subject is acquired among the posture items that are displayed by the posture item display unit; and a display controller that, in response to the input of the input unit, changes a display mode of the posture item of the subject that is displayed by the posture item display unit, such that the display mode indicates that the posture item is input by the input unit.

### CROSS-REFERENCE TO RELATED APPLICATIONS

This application is a continuation of PCT international application Ser. No. PCT/JP2010/62553 filed on Jul. 26, 2010 which designates the United States, incorporated herein by reference, and which claims the benefit of priority from Japanese Patent Applications No. 2009-264276, filed on Nov. 19, 2009, incorporated herein by reference.

### BACKGROUND OF THE INVENTION

1. Field of the Invention

The present invention relates to a medical apparatus system that acquires information on a subject, a capsule medical apparatus system, and a method of displaying a posture item of a subject.

2. Description of the Related Art

In recent years, systems have been proposed in which in-vivo images are taken using a capsule endoscope, data of the taken in-vivo images is received by a receiving device, and the data of the in-vivo images received by the receiving device is displayed on an image display device (see International Publication Pamphlet No. WO 07/077922). In the capsule endoscope system, a doctor observes the in-vivo images that are displayed on the image display device and performs an in-vivo diagnosis on the subject.

### SUMMARY OF THE INVENTION

A medical apparatus system according to an aspect of the present invention includes a medical apparatus that acquires information on a subject; a posture item display unit that displays multiple posture items of the subject in which the medical apparatus acquires the information on the subject; an input unit that inputs a posture item of the subject in which the information on the subject is acquired among the posture items that are displayed by the posture item display unit; and a display controller that, in response to the input of the input unit, changes a display mode of the posture item of the subject that is displayed by the posture item display unit, such that the display mode indicates that the posture item is input by the input unit.

A capsule medical apparatus system according to an aspect of the present invention includes a capsule medical apparatus to be inserted into a subject to take an in-vivo image of the subject; an input unit that inputs information on a posture of the subject in which the in-vivo image is taken; a storage unit that stores the in-vivo image, which is taken by the capsule medical apparatus, in association with the information on the posture of the subject, which is input by the input unit; and an image display unit that displays the in-vivo image, which is stored in the storage unit, and the information on the posture of the subject, which is associated with the in-vivo image.

A method of displaying a posture item of a subject according to an aspect of the present invention includes displaying, on a posture item display unit, multiple posture items of a subject when a medical apparatus acquires information on the subject; and changing, in response to an input of a posture item in which the information on the subject is acquired among the displayed posture items, which is an input made by an input unit, a display mode of the posture item of the subject that is displayed on the posture item display unit, such that the display mode indicates that the posture item is input by the input unit.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram of an entire configuration of a capsule endoscope system of an embodiment of the present invention;

FIG. 2 is a flowchart of a flow of a diagnostic process performed by a control device in FIG. 1;

FIG. 3 is a diagram of an example of a display screen of a posture item;

FIG. 4 is a diagram of an example of a display screen of a posture item;

FIG. 5 is a diagram of an example of a display screen of a posture item;

FIG. 6 is a diagram of an example of a capsule-image display screen;

FIG. 7 is a diagram of a modification of the capsule-image display screen in FIG. 6;

FIG. 8 is a diagram of a modification of the capsule-image display screen in FIG. 6; and

FIG. 9 is a diagram of an example of a diagnostic display screen.

### DETAILED DESCRIPTION OF THE PREFERRED EMBODIMENTS

An entire configuration of a capsule endoscope system as an embodiment of the present invention will be described below with reference to the drawings.

### ENTIRE CONFIGURATION OF CAPSULE ENDOSCOPE SYSTEM

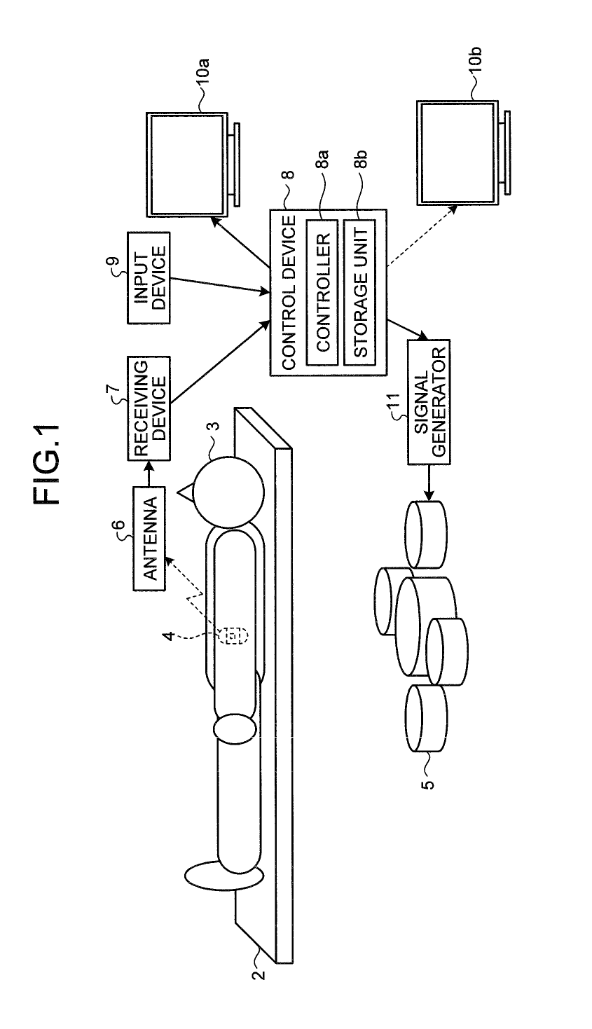

First, an entire configuration of the capsule endoscope system of the embodiment of the present invention will be described with reference to FIG. 1.

FIG. 1 is a schematic diagram of an entire configuration of the capsule endoscope system of the embodiment of the present invention. As illustrated in FIG. 1, a capsule endoscope system 1 of the embodiment of the present invention includes a bed 2; a capsule endoscope 4 serving as a capsule medical apparatus that is inserted together with a liquid, such as water or a normal saline solution, into a subject 3 laid on the bed 2 and then takes in-vivo images of the subject 3; a magnetic field generation device 5 that controls at least any one of the position and the posture of the capsule endoscope 4 that floats in the liquid; a receiving device 7 that receives data of the in-vivo images that are transmitted by radio from the capsule endoscope 4 via an antenna 6; and a control device 8.

The capsule endoscope 4 has an image capturing function of capturing an in-vivo image of a subject and a radio communication function for transmitting by radio various types of information, such as data of in-vivo images. The capsule endoscope 4 is formed to have a size such that it can be easily inserted into the subject 3, and the capsule endoscope 4 has a specific gravity approximately equal to or less than the specific gravity of the liquid, such as water or a normal saline solution. The capsule endoscope 4 sequentially takes in-vivo images at predetermined intervals of, for example, 0.5 second. The capsule endoscope 4 transmits the in-vivo images by radio. In the present embodiment, the capsule endoscope 4 includes imaging devices respectively at the ends of the capsule endoscope 4 in the longitudinal axis direction.

The magnetic field generation device 5 controls at least any one of the position and the posture of the capsule endoscope 4 in the subject 3. Specifically, the magnetic field generation device 5 generates a magnetic field to the capsule endoscope 4, which is inserted into the subject 3, and controls the movement of the capsule endoscope 4 in the liquid by using the magnetic force of the magnetic field. By controlling the movement of the capsule endoscope 4, the magnetic field generation device 5 controls at least any one of the position and the posture of the capsule endoscope 4 in the subject 3. In this case, the capsule endoscope 4 includes a magnet that causes a casing to move according to the magnetic field that is generated by the magnetic field generation device 5.

Operations of the magnetic field generation device are controlled by a signal generator 11 that operates according to control signals from the control device 8. Health care professionals control the movement of the capsule endoscope 4 on the basis of in-vivo images that are taken by one of the two imaging devices arranged in the capsule endoscope 4.

The antenna 6 is realized by using a loop antenna and is arranged on a predetermined position on the body surface of the subject 3. The number of antennas 6 to be arranged is not limited to one and multiple antennas may be arranged. The receiving device 7 receives the data of the in-vivo images, which is transmitted from the capsule endoscope 4 via the antenna 6, and outputs the received data of the in-vivo images to the control device 8. The control device 8 is realized by using a work station. The control device 8 includes a controller 8a and a storage unit 8b.

The controller 8a has a storage function of storing, for example, the data of the in-vivo images received by the receiving device 7, in the storage unit 8b; a display function of displaying, for example, the data of the in-vivo images received by the receiving device 7, on a capsule image display device 10a or a diagnostic display device 10b; a drive control function of outputting, to the signal generator 11, a control signal for controlling driving of the capsule endoscope 4; and an estimation function of estimating the position and the posture of the capsule endoscope 4 on the basis of the generated magnetic field that is estimated from the value of a signal output from the signal generator 11 and from the shape of the magnetic field generator 5.

The storage unit 8b stores the data of the in-vivo images, which are received by the receiving device 7, in association with information on the posture of the subject 3 during the image capturing operation and information on the position and the posture of the capsule endoscope 4 during the image capturing operation. The storage unit 8b also stores, with respect to each in-vivo region of the subject 3, information on posture items of the subject 3 in which observation should be performed and order information that defines the order of the posture items in which observations are performed.

An input device 9, such as a keyboard, a mouse pointer, and a joystick, is connected to the control device 8. By operating the input device 9, the health care professional inputs, to the control device 8, various types of operation input information for, for example, giving an instruction for moving the capsule endoscope 4 and acquiring captured images. The control device 8 functions as a display controller and an operation state confirmation unit according to the present invention. The storage unit 8b functions as a storage unit according to the present invention. The input device 9 functions as an input unit according to the present invention. The capsule image display device 10a functions as a posture item display unit according to the present invention. The diagnostic display device 10b functions as an image display unit according to the present invention.

### DIAGNOSTIC PROCESS

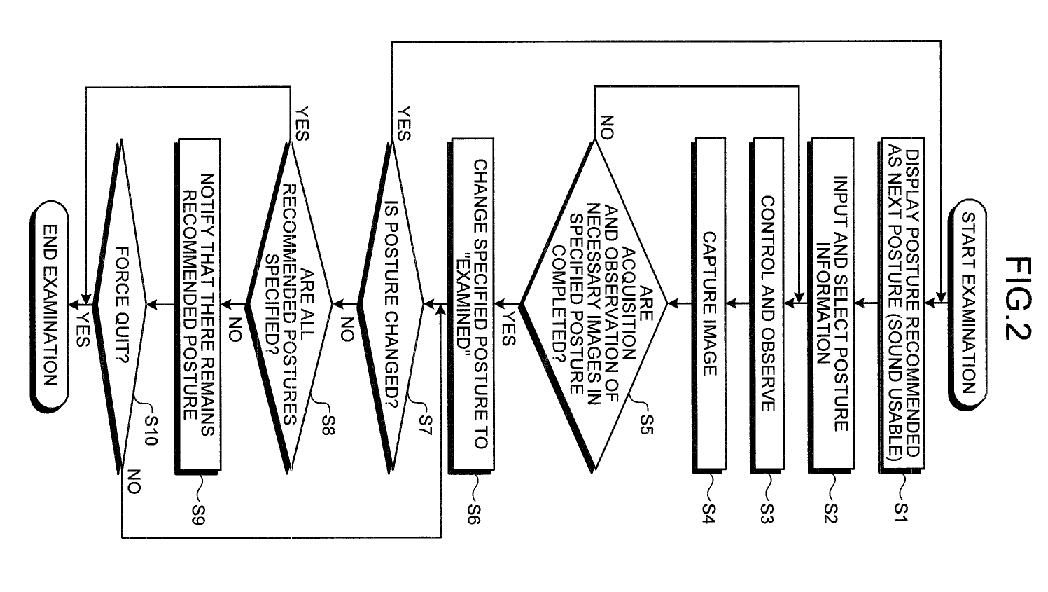

In the capsule endoscope system that has the above configuration, the control device 8 performs the following diagnostic process, which reduces the work of the health care professional required for diagnosis. A flow of the diagnostic process performed by the control device 8 will be described with reference to the flowchart of FIG. 2.

FIG. 2 is a flowchart of a flow of the diagnostic process performed by the control device 8. The flowchart in FIG. 2 starts at a timing when the health care professional gives an instruction for starting the diagnostic process to the control device 8 and the diagnostic process goes to the process at step S1.

In the process at step S1, the control device 10 displays recommended posture items of the subject 3. When the process is after the posture of the subject 3 is changed, the control device 10 notifies a posture item that is not stored as “Examined” among the posture items that are displayed in the previous process. Specifically, the control device 8 notifies a posture item that is not stored as “Examined” by, on the basis of posture information that defines the order of posture items in which observations should be performed, changing the display color of the posture item in which observation should be performed next among the posture items in FIG. 4 or informing a posture in which observation should be performed next using a pop-up display or an audio output saying “Next is the Decubitus right”. Accordingly, the process at step S1 is completed and the diagnostic process goes to the process at step S2.

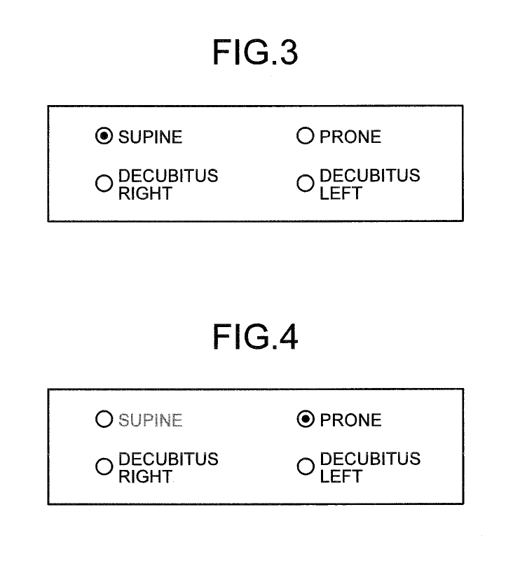

In the process at step S2, the control device 8 operates the input device 9 to cause it to input the information on the posture of the subject 3 in which in-vivo images are to be observed. Specifically, the control device 8 displays, for example, as illustrated in FIG. 3, four posture items “Supine”, “Prone”, “Decubitus right”, and “Decubitus left” on the capsule image display device 10a as the information on recommended postures of the subject 3. The control device 8 then operates the input device 9 to click a check box provided to each posture item, thereby inputting the information on the posture of the subject 3 in which observation is performed. The control device 8 promotes the diagnostic process to the process at step S3 in the timing when the input device 9 inputs the posture information on the subject 3.

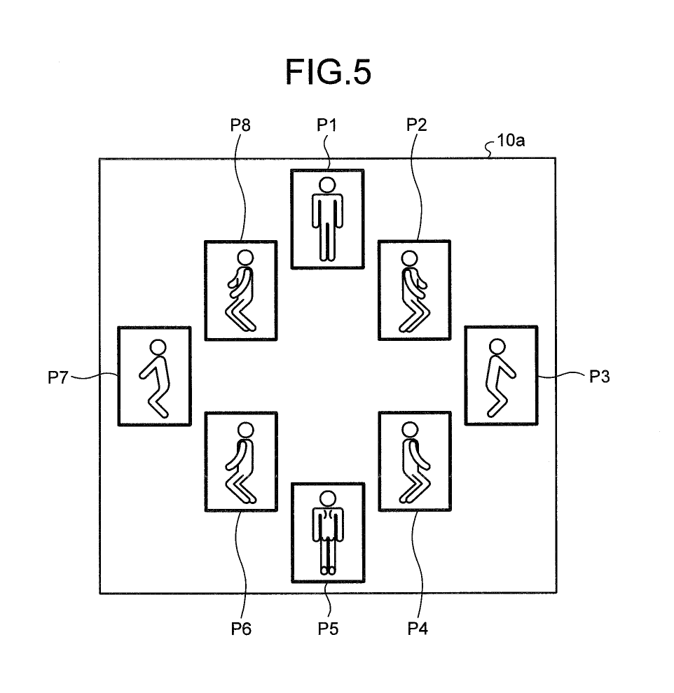

As illustrated in FIG. 5, the control device 8 may inform the recommended posture items of the subject 3 by displaying images P1 to P8 that schematically show the posture items of the subject 3. In FIG. 5, the image P1 is an image showing the posture item “Supine”, the image P2 is an image showing a posture item between the posture item “Supine” and the posture item “Decubitus left”, the image P3 is an image showing the posture item “Decubitus left”, the image P4 is an image showing a posture item between the posture item “Decubitus left” and the “Prone”, the image P5 is an image showing the posture item “Prone”, the image P6 is an image showing a posture item between the posture item “Prone” and the posture item “Decubitus righ”, the image P7 is an image showing the posture item “Decubitus right”, and the image P8 is an image showing a posture item between the posture item “Decubitus right” and the posture item “Supine”.

In the image display example in FIG. 5, the control device 8 may, at the timing when the input device 9 inputs a posture item of the subject 3, change the display color of the image corresponding to the input posture item or display a name of the posture item near the image corresponding to the input posture item. Because of such a process, the input posture item can be easily recognized and a posture item can be input accurately. The control device 8 may, at the timing when the input device 9 selects an image, display the name of the posture item shown by the selected image.

When a posture item is selected, the control device 8 displays “Under examination” for the selected posture item. When a posture item is selected next, the control device 8 displays “Examined” for the already selected posture item and displays “Under examination” for the newly selected posture item.

In the process at step S3, in response to an operation of the input device 9, the control device 8 operates to guide the capsule endoscope 4 and to take in-vivo images and sequentially stores the taken in-vivo images in the storage unit 8b. Here, the control device 8 stores, in the storage unit 8b, the taken in-vivo images in association with information on the posture of the subject 3 during the image capturing operation and information on the position and the posture of the capsule endoscope 4 during the image capturing operation. Accordingly, the process at step S3 is completed and the diagnostic process goes to the process at step S4.

In the process at step S4, in response to an operation of the input device 9, the control device 8 captures the in-vivo images and stores the captured in-vivo images in the storage unit 8b as captured images. Here, the control device 8 stores, in the storage unit 8b, the captured images in association with the information on the posture of the subject 3 in which the captured images are taken and information on the position and the posture of the capsule endoscope 4 in which the captured images are taken. In the present embodiment, a posture item of the subject 3 in which observation is performed is input in the process at step S2. Alternatively, a posture item of the subject 3 may be input when an instruction for acquiring a captured image is given. Note that, in this case, only the information on the posture of the subject 3 in which the captured images are acquired is stored. Accordingly, the process at step S4 is completed and the diagnostic process goes to the process at step S5.

In the process at step S5, according to an operation of the input device 9, the control device 8 determines whether acquisition and observation of necessary in-vivo images are completed. When the result of the determination indicates that acquisition and observation of necessary in-vivo images are not completed, the control device 8 returns the diagnostic process to the process at step S3. In contrast, when acquisition and observation of necessary in-vivo images are completed, the control device 8 promotes the diagnostic process to the process at step S6. When the observation time in the same posture lasts for a predetermined time or more, the control device 8 may inform the information on the posture of the subject 3.

In the process at step S6, the control device 8 stores, as “Examined”, the posture item of the subject 3 in which acquisition and observation of necessary in-vivo images are completed. Specifically, in the case where, for example, as illustrated in FIG. 3, the four posture items “Supine”, “Prone”, “Decubitus right”, and “Decubitus left” are displayed as posture items of the subject 3 in which observation should be performed, when acquisition and observation of in-vivo images of the subject 3 whose posture is “Supine” are completed, the control device 8 changes the display mode of the posture item of the subject 3 that is stored as “Examined” by, for example, displaying “Supine” in a lighter display color as illustrated in FIG. 4. Because of such a process, the posture item in which acquisition and observation of necessary in-vivo images are completed can be visually confirmed. Accordingly, in all the posture items necessary for in-vivo regions to be observed, in-vivo images can be acquired and observed without omission. Accordingly, the process at step S6 is completed and the diagnostic process goes to the process at step S7.

In the process at step S7, according to an operation of the input device 9, the control device 8 determines whether the posture of the subject 3 is changed. A detection means, such as a weight sensor, may be arranged under the bed 2 to detect whether the posture of the subject 3 is changed. When the result of the determination indicates that the posture of the subject 3 is changed, the control device 8 returns the diagnostic process to the process at step S1. When the posture of the subject 3 is not changed, the control device 8 promotes the diagnostic process to the process at step S8. It is desirable that, when the posture of the subject 3 is changed, the control device 8 displays a display for confirming whether the information on the posture item, which is input in the process at step S2, is automatically reset or the posture item is reset. Such a process helps inputting a posture item again when the posture is changed and accordingly in-vivo images are taken according to an appropriate posture item.

In the process at step S8, the control device 8 determines whether all the posture items that are displayed in the process at step S1 are stored as “Examined”. When the result of the determination indicates that all the posture items that are displayed in the process at step S1 are stored as “Examined”, the control device 8 ends the diagnostic process. In contrast, when all the posture items that are displayed in the process at step S1 are not stored as “Examined”, the control device 8 promotes the diagnostic process to the process at step S9.

In the process at step S9, the control device 8 notifies that there remains a posture item that is not stored as “Examined”. Specifically, the control device 8 notifies that there remains a posture item that is not stored as “Examined” by changing the display color of the posture item that is not stored as “Examined” or informing that there is a recommended posture item by saying, for example, “An recommended posture item remains” using a pop-up display or an audio output. Accordingly, the process at step S9 is completed and the diagnostic process goes to step S10.

In the process at step S10, according to an operation of the input device 9, the control device 8 determines whether an instruction for ending the diagnostic process is given. When the result of determination indicates that an instruction for ending the diagnostic process is not given, the control device 8 returns the diagnostic process to the process at step S7. In contrast, when an instruction for ending the diagnostic process is given, the control device 8 ends the diagnostic process. If an instruction for ending the diagnostic process is given when acquisition and observation of in-vivo images are not completed in all the posture items that are displayed in the process at step S1, the control device 8 may give information that examinations in all the posture items are not completed.

### CONFIGURATION OF CAPSULE-IMAGE DISPLAY SCREEN

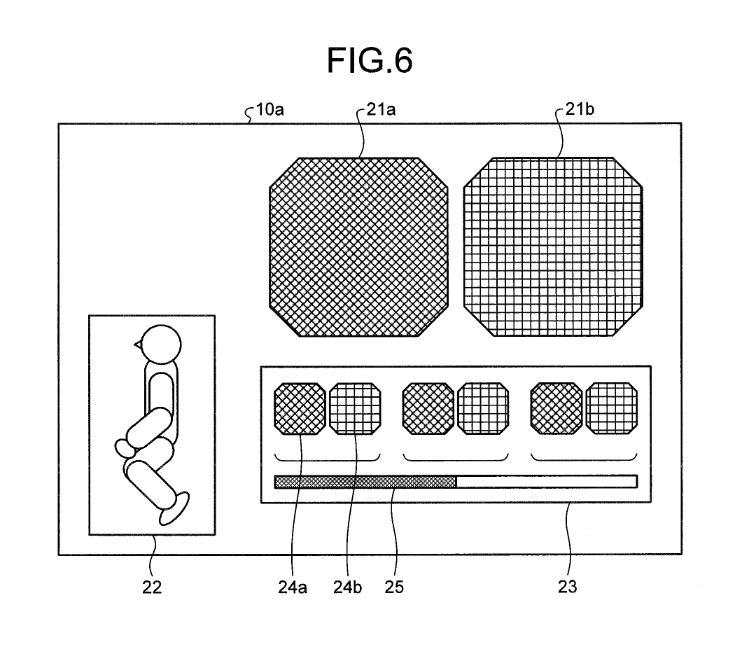

Next, a configuration of the capsule-image display screen that is displayed on the capsule image display device 10a in the above-described diagnostic process will be described with reference to FIG. 6.

FIG. 6 is a schematic diagram of a configuration of a capsule-image display screen that is displayed on the capsule image display device 10a. As illustrated in FIG. 6, the capsule-image display screen that is displayed on the capsule image display device 10a includes capsule image display areas 21a and 21b, a posture information display area 22, and a captured-image display area 23. The capsule image display areas 21a and 21b display in-vivo images that are taken by the two imaging devices of the capsule endoscope 4 in the process at step S2. The posture information display area 22 schematically displays a posture item that is associated with the in-vivo images displayed in the capsule image display areas 21a and 21b, i.e., the posture item of the subject 3 in which the in-vivo images that are displayed in the capsule image display areas 21a and 21b are taken. The captured-image display area 23 includes multiple captured images 24a and multiple captured images 24b that are captured by the two imaging devices of the capsule endoscope 4 and a scroll bar 25 that scrolls and displays the captured images 24a and 24b. Such a capsule-image display screen allows a real-time visual confirmation about in which posture the in-vivo images are currently taken. In the capsule-image display screen in FIG. 6, the in-vivo images that are taken by the two imaging device of the capsule endoscope 4 are displayed in the same size. Alternatively, an in-vivo image that is taken by a specified imaging device may be displayed in a size larger than an image taken by the other imaging device or an in-vivo image taken by the not-specified imaging device may not be displayed.

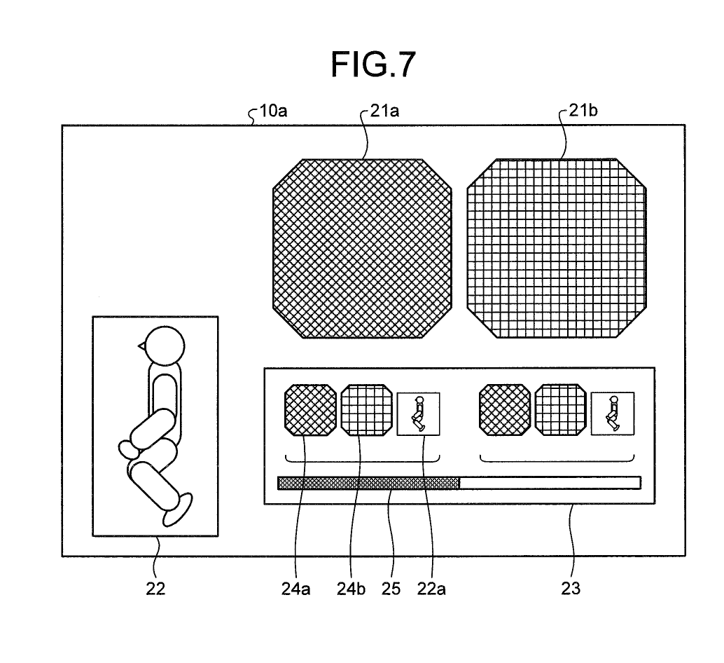

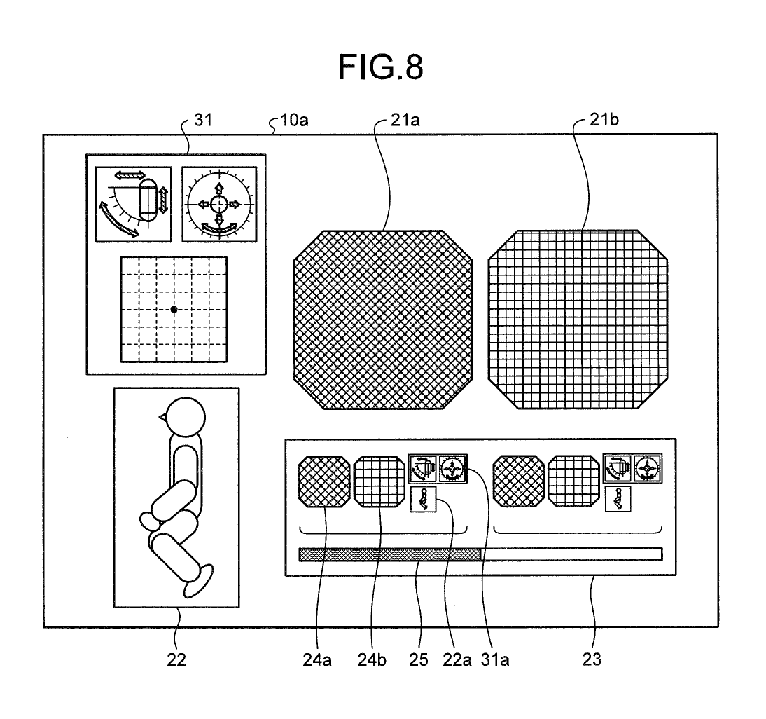

On the capsule-image display screen in FIG. 6, only the posture item of the subject 3 corresponding to the in-vivo images taken by the capsule endoscope 4 is displayed. Alternatively, as illustrated in FIG. 7, the posture item of the subject 3 in which the captured images are taken may be displayed in a posture information display area 22a. Alternatively, as illustrate in FIG. 8, the information on the movement of the capsule endoscope 4 may be displayed on operation information display areas 31 and 31a. In the example of FIG. 8, both of the posture item of the subject 3 and the information on guidance of the capsule endoscope 4 are displayed. Alternatively, only the operation information on the capsule endoscope 4 may be displayed. Information on an estimated position and an estimated posture of the capsule endoscope 4, an operation amount of the input device 9, an imaging device that is specified in the image taking operation, or an operation mode of the capsule endoscope 4 (for example, a mode of operating in liquid or a high-speed operation mode) can be taken as examples of the operation information on the capsule endoscope 4.

### CONFIGURATION OF DIAGNOSTIC DISPLAY SCREEN

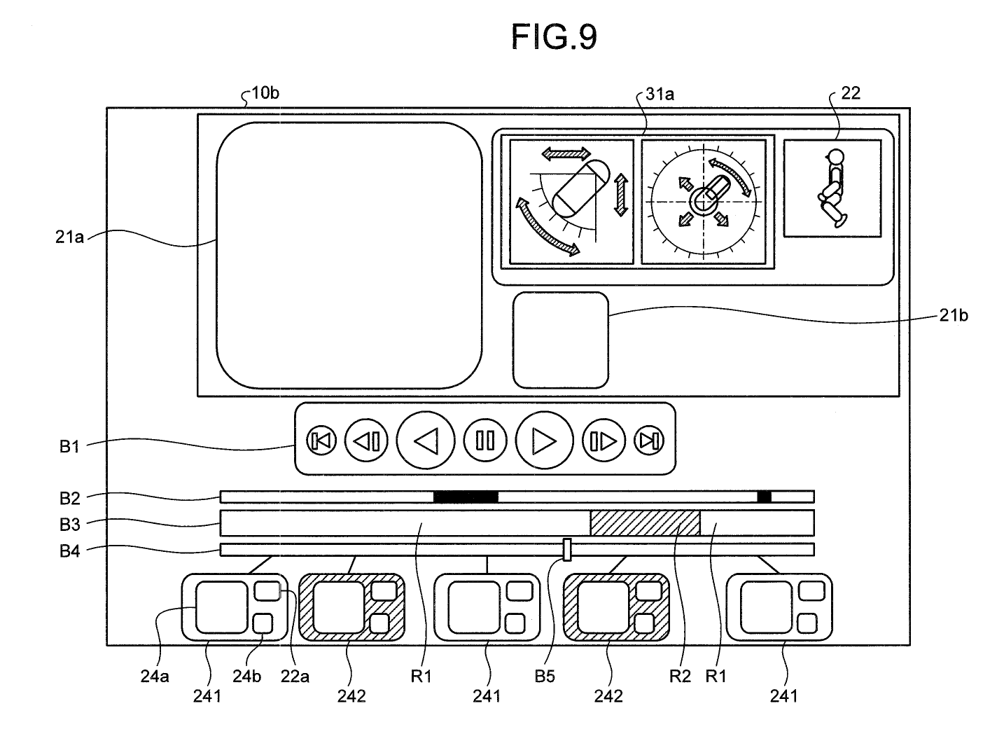

Lastly, with reference to FIG. 9, a configuration will be described of a diagnostic display screen that is displayed on the diagnostic display device 10b when an in-vivo diagnosis is performed on the subject on the basis of the in-vivo images obtained by the above-described diagnostic process.

FIG. 9 is a schematic diagram of a configuration of the diagnostic display screen that is displayed on the diagnostic display device 10b. As illustrated in FIG. 9, the diagnostic display screen includes the capsule image display areas 21a and 21b, the posture information display area 22, the captured-image display area 23, the operation information display area 31a, a play button B1, a red bar B2, an average color bar B3, and a time bar B4. The capsule image display areas 21a and 21b, the posture information display area 22, the captured-image display area 23, and the operation information display area 31 have the same configurations as those of the capsule-image display screen, which is described above, and thus descriptions thereof will be omitted.

The play button B1 is a manipulator for sequentially displaying in-vivo images that are taken in the diagnostic process. The play button B1 is provided with an orderly play button, a reverse play button, and a stop button. The red bar B2 indicates a ratio of red in an in-vivo image that is taken by the capsule endoscope 4. On the basis of the ratio of red, it can be determined whether a bleeding occurs in an observed region. The average color bar B3 indicates the average color of the in-vivo image taken by the capsule endoscope 4. On the basis of the display color of the average color bar B3, it can be determined whether the observed region or the imaging device is changed. Specifically, in the example of FIG. 9, the display color differs between the region R1 and the region R2 of the average color bar B3. Thus, it is known that the imaging device used for taking the image of the region R1 is different from the imaging device used for taking the image of the region R2, e.g., the imaging device used for taking the image of the region R2 is not the specified imaging device. The time bar B4 contains the length of time required for the diagnostic process. The time at which the displayed in-vivo image is taken can be confirmed on the basis of the position of the button B5 in the time bar B4. In the present embodiment, the captured images 24a and 24b and the posture item 22a are displayed in association with the time, on the time bar B4, at which the captured images are captured. In addition, captured images 241 that are specified in a diagnosis and captured images 242 that are specified in a guidance operation are displayed separately. By using such a diagnostic display screen, a doctor can easily confirm in which posture an in-vivo image is captured.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical apparatus system comprising:
a medical apparatus that acquires information on a subject;
a posture item display unit that displays multiple posture items of the subject in which the medical apparatus acquires the information on the subject;
an input unit that inputs a posture item of the subject in which the information on the subject is acquired among the posture items that are displayed by the posture item display unit; and
a display controller that, in response to the input of the input unit, changes a display mode of the posture item of the subject that is displayed by the posture item display unit, such that the display mode indicates that the posture item is input by the input unit.

2. The medical apparatus system according to claim 1, wherein, when the input unit inputs a posture item of the subject in which acquisition of the information is started, the display controller changes the display mode of the posture item.

3. The medical apparatus system according to claim 2, wherein, when the input unit inputs information indicating completion of the acquisition of the information in the posture item, the display controller further changes the changed display mode of the posture item.

4. The medical apparatus system according to claim 1, wherein
the display controller includes recommended-posture information that defines posture items in which acquisition of the information is recommended, and
the display controller changes the display mode of the posture item, which is displayed by the posture item display unit, on the basis of the recommended-posture information.

5. The medical apparatus system according to claim 4, wherein the display controller changes the recommended-posture information in accordance with a region of the subject from which the information is acquired.

6. The medical apparatus system according to claim 4, wherein
the recommended-posture information contains information on an order of the posture items, and
the display controller changes the display mode of the posture item, which is displayed by the posture item display unit, on the basis of the information on the order.

7. The medical apparatus system according to claim 6, wherein the display controller changes the display mode of a posture item of the subject in which information is acquired next among the posture items, which are displayed by the posture item display unit, on the basis of the recommended-posture information.

8. The medical apparatus system according to claim 1, wherein the medical apparatus is a capsule medical apparatus to be inserted into the subject to acquire image information.

9. A capsule medical apparatus system comprising:
a capsule medical apparatus to be inserted into a subject to take an in-vivo image of the subject;
an input unit that inputs information on a posture of the subject in which the in-vivo image is taken;
a storage unit that stores the in-vivo image, which is taken by the capsule medical apparatus, in association with the information on the posture of the subject, which is input by the input unit; and
an image display unit that displays the in-vivo image, which is stored in the storage unit, and the information on the posture of the subject, which is associated with the in-vivo image.

10. The capsule medical apparatus system according to claim 9, wherein
the image display unit has a capture function, and
the image display unit includes a captured-image display area in which an in-vivo image that is taken by using the capture function and posture information, which is associated with the in-vivo image.

11. The capsule medical apparatus system according to claim 9, further comprising an operation state confirmation unit that confirms information on operation state of the capsule medical apparatus, wherein
the storage unit stores the information on the operation state of the capsule medical apparatus, which is confirmed by the operation state confirmation unit, in association with the in-vivo image, and
the image display unit displays the in-vivo image, which is stored in the storage unit, and the information on the operation state of the capsule medical apparatus, which is associated with the in-vivo image.

12. The capsule medical apparatus system according to claim 10, further comprising an operation state confirmation unit that confirms information on operation state of the capsule medical apparatus,
wherein the image display unit displays the information on the operation state of the capsule medical apparatus, which is confirmed by the operation state confirmation unit, in the captured-image display area.

13. The capsule medical apparatus system according to claim 11, wherein
the capsule medical apparatus includes at least one magnetic material, and
the capsule medical apparatus system further comprises a guidance magnetic field generation device that acts on the at least one magnetic material to guide the capsule medical apparatus,
wherein the operation state confirmation unit confirms, as the information on the operation state of the capsule medical apparatus, information on a magnetic field generated by the guidance magnetic field generation device.

14. A method of displaying a posture item of a subject, comprising:
displaying, on a posture item display unit, multiple posture items of a subject when a medical apparatus acquires information on the subject; and
changing, in response to an input of a posture item in which the information on the subject is acquired among the displayed posture items, which is an input made by an input unit, a display mode of the posture item of the subject that is displayed on the posture item display unit, such that the display mode indicates that the posture item is input by the input unit.

15. The method according to claim 14, further comprising:
determining whether all necessary information on the displayed posture items of the subject is acquired; and
changing a display mode of a posture item regarding which the determining determines that all necessary information is acquired.

16. The method according to claim 14, further comprising comparing information on the posture of the subject, on which the change is made, with pre-stored recommended-posture information that defines recommended posture items of the subject.

17. The method according to claim 16, further comprising changing, when a result of the comparing indicates that display modes of all the recommended-posture information on the subject are not changed, the posture information on the subject of which display mode is not changed to a display that is different from that of the posture information on the subject of which display mode is changed.

18. The display method according to claim 16, further comprising informing, when a result of the comparing indicates that display modes of all the recommended-posture information on the subject are not changed, that there is posture information on the subject of which display mode is not changed.

19. A medical apparatus system comprising:
a medical apparatus that acquires information on a subject;
a posture item display means for displaying multiple posture items of the subject in which the medical apparatus acquires the information on the subject;
an input means for inputting a posture item of the subject in which the information on the subject is acquired among the posture items that are displayed by the posture item display means; and
a display control means for, in response to the input of the input means, changing a display mode of the posture item of the subject that is displayed by the posture item display means, such that the display mode indicates that the posture item is input by the input means.

20. A capsule medical apparatus system comprising:
a capsule medical apparatus to be inserted into a subject to take an in-vivo image of the subject;
an input means for inputting information on a posture of the subject in which the in-vivo image is taken;
a storage means for storing the in-vivo image, which is taken by the capsule medical apparatus, in association with the information on the posture of the subject, which is input by the input means; and
an image display means for displaying the in-vivo image, which is stored in the storage means, and the information on the posture of the subject, which is associated with the in-vivo image.
